(19) United States
(12) Patent Application Publication
(10) Pub. No.: US 2016/0030685 A1
(43) Pub. Date: Feb. 4, 2016

(54) DEVICE FOR REDUCTION OF NEEDLE INSERTION SITE PAIN IN PATIENTS

(71) Applicant: Lane Biomedical, LLC, Columbus, GA (US)

(72) Inventor: Joshua Edward Lane, Columbus, GA (US)

(21) Appl. No.: 14/752,310

(22) Filed: Jun. 26, 2015

Related U.S. Application Data
(60) Provisional application No. 62/031,576, filed on Jul. 31, 2014.

(51) Int. Cl. A61M 5/42; A61F 7/02

(52) U.S. Cl. CPC A61M 5/422; A61F 7/02; A61F 2007/0052

(57) ABSTRACT

Various embodiments of the present invention are directed to a device for the reduction of needle insertion site pain in patients. Various embodiments comprise a shell defining a hollow interior cavity filled at least in part by a freezable substance. The shell defines a first surface and a second surface opposite the first surface, and a passage extending through the shell between the first surface and the second surface, wherein the passage is configured to at least partially surround a needle insertion site while a needle is inserted therethrough. At least one of the first and second surfaces comprise a plurality of protrusions distributed therein and configured to be depressed into the skin of the patient. When chilled and depressed against the patient's skin, the freezable device provides a sensory confusion sensation to the patient, thereby reducing perceived pain caused by the needle insertion.

### CROSS-REFERENCE TO RELATED APPLICATIONS

This patent application is claims priority to Provisional Application Ser. No. 62/031,576 entitled “Device for Reduction of Needle Insertion Site Pain in Patients,” filed Jul. 31, 2014, which is incorporated herein by reference in its entirety.

### BACKGROUND

In an effort to reduce the perceived pain and the general unpleasantness of needle pricks associated with medical shots and blood testing, devices and methods may be used to distract patients from pain associated with a needle insertion. Such distraction may be accomplished by having the patient focus on an unrelated stimulant (e.g., a conversation with a third-party) or by confusing the nerves around the needle insertion site.

Various devices have been developed for confusing the nerves around a needle insertion site. However, many of these devices require power sources (e.g., batteries) that must be replaced or charged periodically. Other existing devices utilize only a single method of confusing nerves around the pain site, and therefore may not maximize potential pain relief for a patient. Moreover, existing devices are generally configured to confuse nerves on a single side of an insertion site, and therefore these devices may be difficult to align in an appropriate location to maximize pain reduction.

Therefore a need exists for an unpowered device capable of effectively reducing pain caused by needle insertion that may be easily aligned around the needle insertion site to maximize pain reduction associated with needle insertion.

### BRIEF SUMMARY

Various embodiments are directed to a freezable device for reducing pain perception during insertion of a needle into a patient, comprising a shell having a first surface and a second surface opposite the first surface and comprising a pliable, rigid, and/or semi rigid material; and at least one, and potentially two or more, passage extending through the device from the first surface to the second surface, wherein the passage is configured to at least partially surround a needle insertion site on the patient; wherein at least one of the first surface and second surface comprise a plurality of protrusions having a conical and/or pyramid shape and configured to be pressed against the patient; and wherein the shell defines an interior cavity, the inner cavity being filled, at least in part, with a first substance, such as a gel. In various embodiments, the shell may be sterile for use in a professional medical environment. Moreover, in various embodiments, the shell comprises a perimeter surface surrounding the shell and extending between the first surface and the second surface to define the interior cavity of the shell; and wherein the passage defines a passage surface at least partially surrounding the passage and extending between the first surface and the second surface; and wherein the perimeter surface comprises the passage surface. Moreover, the passage may define a first hole extending through the first surface and a second hole extending through the second surface, and a passage perimeter surface extending between the first hole and the second hole. In various embodiments, the freezable device may additionally comprise a sterile insert defined as a hollow cylinder having open ends, to be placed within the passage.

Various embodiments are directed to a freezable device for reducing pain perception during insertion of a needle into a patient, comprising: a shell having a first surface and a second surface opposite the first surface; and at least one, and potentially two or more, passage extending through the device from the first surface to the second surface, wherein the passage is configured to at least partially surround a needle insertion site on the patient and wherein a membrane covers at least one end of the passage; wherein at least one of the first surface and second surface comprise a plurality of protrusions configured to be pressed against the patient; and wherein the shell defines an interior cavity, the inner cavity being filled, at least in part, with a first substance. The shell and the membrane may be sterile for use in a professional medical environment. The membrane may be configured to be punctured by a needle.

Moreover, various embodiments are directed to a method of reducing perceived pain during a needle insertion into a patient, the method comprising steps for: reducing the temperature of a freezable device, the freezable device comprising a first surface and a second surface opposite the first surface, and a passage extending through the freezable device between the first surface and the second surface, and wherein at least one of the first surface and the second surface comprise a plurality of protrusions; pressing the plurality of protrusions against the patient's skin such that a needle may be inserted through the passage and into the patient's skin at a needle insertion location; and inserting the needle through the passage and into the patient's skin at the needle insertion location. Various embodiments additionally comprise steps for sterilizing the freezable device prior to pressing the freezable device against the patient's skin. Moreover, various embodiments additionally comprise steps for sterilizing an insert and/or inserting the insert into the passage prior to pressing the freezable device against the patient's skin. Moreover, in various embodiments, a membrane covers at least one end of the passage, and the method additionally comprises puncturing the membrane with the needle.

### BRIEF DESCRIPTION OF THE DRAWINGS

Reference will now be made to the accompanying drawings, which are not necessarily drawn to scale, and wherein:

FIG. 1 is a top-perspective view of a freezable device according to various embodiments of the present invention;

FIG. 2 is a side perspective view of a freezable device according to various embodiments of the present invention;

FIG. 3 is a side view of a freezable device according to various embodiments of the present invention;

FIG. 4 is a bottom view of a freezable device according to various embodiments of the present invention;

FIGS. 5A-5C are bottom views of freezable devices according to various embodiments of the present invention; and

FIG. 6 is a perspective view of a freezable device in use according to various embodiments of the present invention.

### DETAILED DESCRIPTION

The present invention will now be described more fully hereinafter with reference to the accompanying figures, in which some, but not all embodiments of the invention are shown. Indeed, the invention may be embodied in many different forms and should not be construed as limited to the embodiments set forth herein. Rather, these embodiments are provided so that this disclosure will satisfy applicable legal requirements.

Overview

A device for reducing perceived pain associated with a needle insertion by confusing nerves surrounding a needle insertion site is provided. In various embodiments, the device may comprise a freezable device having one or more passages extending therethrough and having a plurality of raised protrusions on an exterior surface of the freezable device. In various embodiments, the freezable device may comprise a pliable exterior shell having the plurality of raised protrusions on the exterior surface of the shell. However, in various embodiments the freezable device may comprise a rigid and/or semi-rigid exterior shell. The exterior shell may define an interior cavity filled at least partially with a freezable substance (e.g., a freezable liquid and/or a freezable gel). Alternatively, the interior cavity may be filled with a gel configured to maintain a gelatinous state at low temperatures. As a non-limiting example, the gel may be configured to maintain a gelatinous state at 0 degrees Fahrenheit, such that the freezable device may maintain flexibility when subjected to low temperatures. As yet another alternative, the interior cavity may be filled with a plurality of small solid and/or gelatinous objects (e.g., pellets) such that the freezable device maintains flexibility at low temperatures. In various embodiments, the freezable device may be sufficiently flexible to be used in anatomic areas with variable topography (e.g., near a patient's nose) at low temperatures.

In various embodiments, the freezable device may be sterilized prior to use. Specifically, a plurality of sterile freezable devices may be stored within a dispenser in a freezer and may be removed as needed. In various embodiments, the freezable device may be configured to be used for a single needle insertion prior to disposal.

Various embodiments provide a method of using a freezable device for reducing perceived pain associated with a needle insertion. In various embodiments, the method may comprise steps for freezing a freezable device having an exterior surface including a plurality of protrusions, one or more passages extending through the freezable device, and an interior cavity filled with a gel or freezable liquid. After chilling the freezable device, an insert may be placed within at least one of the one or more passages extending through the thickness of the freezable device. Following placement of the insert, the freezable device may be placed over a needle insertion site such that a needle may be inserted through the passage and into a patient's skin at the needle insertion site. The freezable device may be pressed firmly into the patient's skin prior to insertion of the needle. The needle may then be inserted through the passage and into the patient's skin. In various embodiments, a user may proceed with placement of the freezable device and insertion of the needle without placing an insert within one of the one or more passages.

Freezable Device

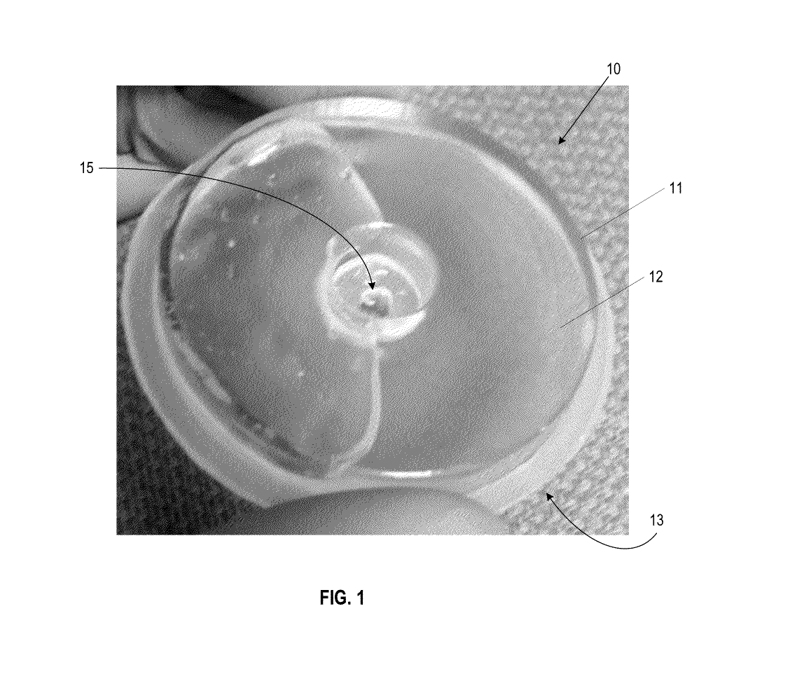

FIG. 1 is a top perspective view of a freezable device 10 according to various embodiments of the present invention. As illustrated in FIG. 1, the freezable device 10 comprises a shell 11 defining a hollow interior cavity. In various embodiments, the shell 11 comprises a pliable material (e.g., a plastic material, rubber material, and/or the like), such that the freezable device 10 may conform to anatomical features of a patient to reduce needle site insertion pain at a plurality of locations on a patient's body. For example, the freezable device 10 may be configured to conform to the bridge of a patient's nose, the area adjacent a patient's eye socket, and/or the like. Moreover, the pliable material may be configured to maintain flexibility at the low temperatures typical of a residential, commercial, or medical freezer (e.g., temperatures between −30 degrees and 10 degrees Fahrenheit, and more preferably temperatures between −15 degrees and 0 degrees Fahrenheit).

Moreover, in various embodiments, the hollow interior cavity may be filled at least in part with a freezable substance (e.g., a freezable liquid and/or a freezable gel). For example, the hollow interior cavity may be filled at least in part with water. Alternatively, the interior cavity may be filled with a gel configured to maintain a gelatinous state at low temperatures. As a non-limiting example, the gel may be configured to maintain a gelatinous state at 0 degrees Fahrenheit, such that the freezable device 10 may maintain flexibility when subjected to low temperatures. As yet another alternative, the interior cavity may be filled with a plurality of small solid and/or gelatinous objects (e.g., pellets) such that the freezable device 10 maintains flexibility at low temperatures.

In various embodiments, the shell 11 may comprise a rigid and/or semi-rigid material (e.g., a hard plastic, metal, and/or the like).

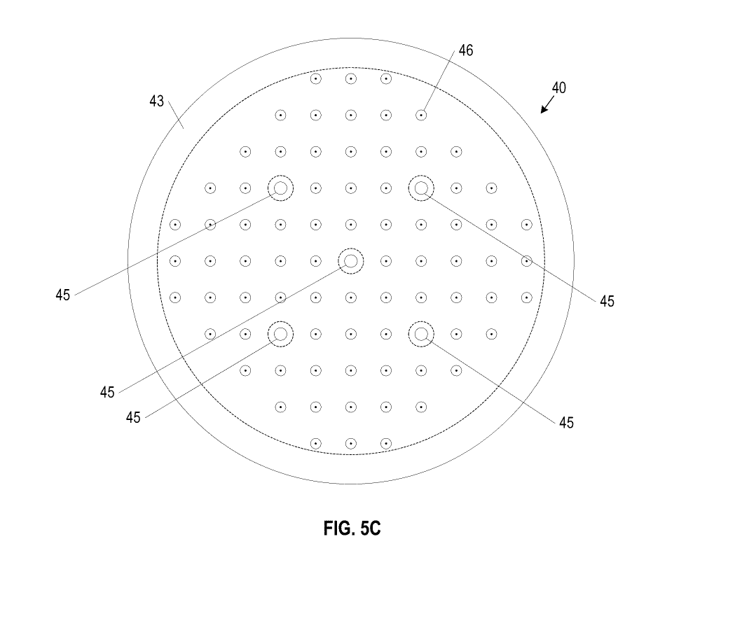

As shown in FIG. 1, the shell 11 may have a circular shape, although the shell 11 may have any of a variety of other shapes, such as a square shape, a triangular shape, and/or the like (as discussed in reference to FIGS. 5A and 5B herein). The shell 11 may have any of a variety of sizes. As non-limiting examples the shell 11 may have a 1-inch outer diameter or a 2-inch outer diameter. Moreover, the shell 11 may define a first surface 12 and a second surface 13 opposite the first surface.

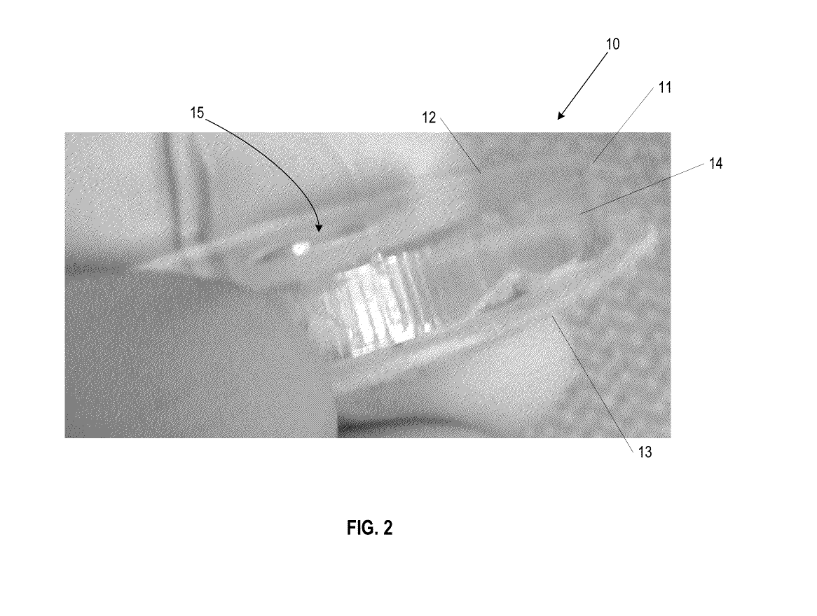

Referring briefly to FIG. 2, the shell 11 may additionally define a perimeter surface 14 extended around the perimeter of the shell and extending between the first surface 12 and the second surface 13.

Moreover, referring again to FIG. 1, the freezable device 10 may have a passage 15 extending therethrough, between the first surface 12 and the second surface 13. As shown in FIG. 1, the passage 15 may comprise a hole (e.g., a circular hole) extending through the entirety of the shell 11 such that the hole extends through the first surface 12 and the second surface 13. In various embodiments, the passage 15 may define a substantially cylindrical hole extending through the freezable device 10. As shown in FIG. 1, the shell 11 may be enclosed such that the hollow interior cavity is sealed, and thus the passage 15 may define a passage surface extending around the perimeter of the passage and between the first surface 12 and the second surface 13.

In various embodiments, one or more membranes may cover one or both ends of the passage 15, such that the membrane may be punctured by a needle when inserted through the passage 15. As a non-limiting example, the one or more membranes may be defined as a portion of the first surface 12 and/or the second surface 13 extending across the passage 15.

As illustrated in FIGS. 1-4, the freezable device 10 may have a thin profile, such that the thickness of the freezable device 10 is substantially smaller than the length, width, and/or diameter of the freezable device 10. The one or more passages 15 may extend through the freezable device 10 parallel to the thickness, such that the freezable device 10 maintains a large surface area that may be placed in contact with a patient's skin during insertion of a needle through one of the one or more passages 15 into the patient's skin. As shown in FIG. 1, the one or more of the passages 15 may extend through the thickness of the freezable device 10 at the center of the freezable device, although one or more of the passages 15 may extend through the thickness of the freezable device at any location.

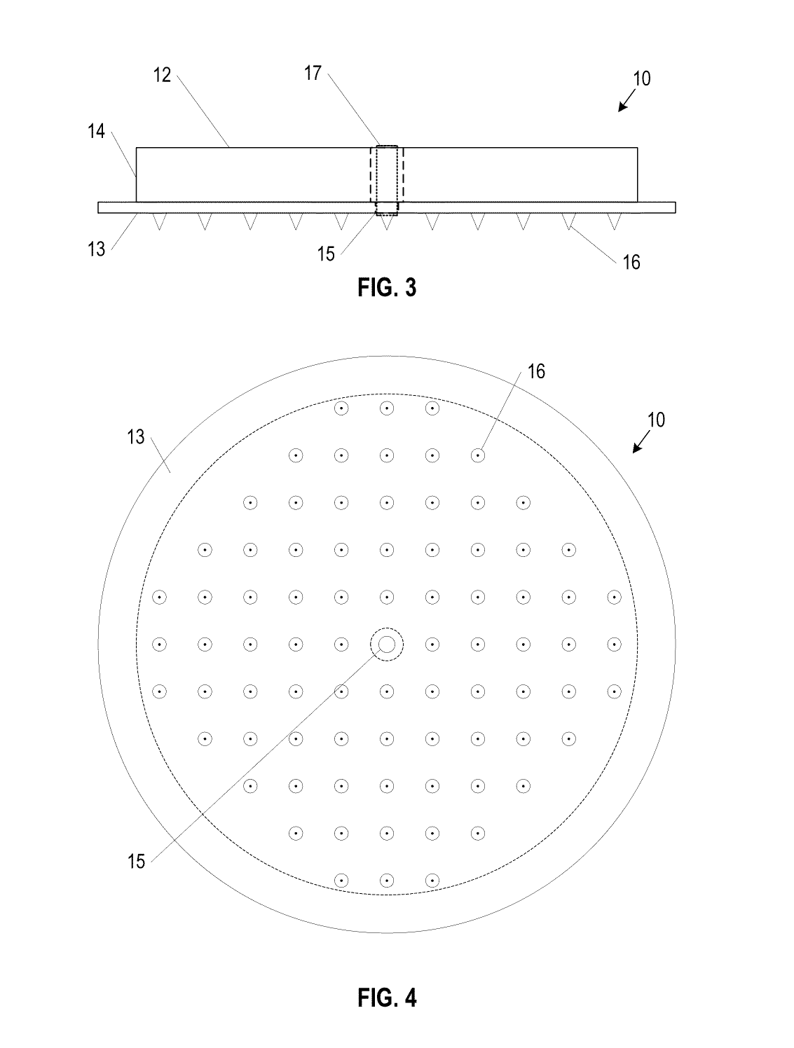

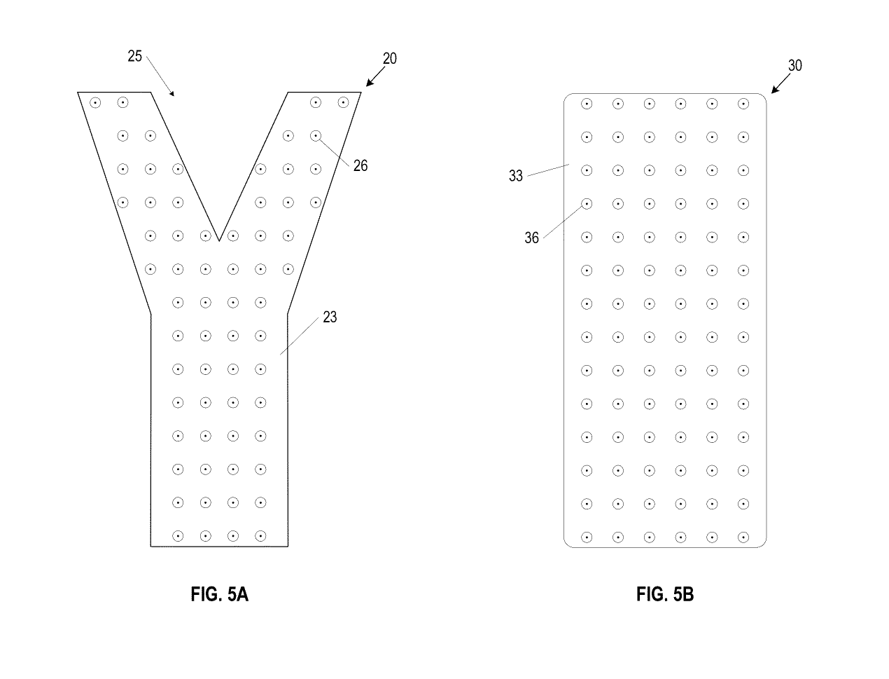

Various embodiments additionally comprise a plurality of protrusions on at least one of the first surface 12 and the second surface 13. As shown in FIGS. 3 and 4, which are schematic side- and bottom-views of a freezable device 10, respectively, the plurality of protrusions 16 may be distributed across the second surface 13. In various embodiments, the various protrusions 16 may comprise conical protrusions, although the protrusions may comprise any of a variety of other shapes (e.g., pyramid shaped, frustum shaped, trapezoidal, hemispherical, cubical, cylindrical, and/or the like). In various embodiments having conical or pyramid shaped protrusions, each protrusion 16 may have a dull point, such that firmly pressing the plurality of protrusions into a patient's skin will not cause the patient to experience pain. As illustrated in FIGS. 3 and 4, the plurality of protrusions 16 may be spaced at least substantially uniformly across the first surface 12 and/or the second surface 13.

Moreover, as shown in FIG. 3, the freezable device 10 may additionally comprise one or more inserts 17 configured to be inserted into one of the one or more passages 15. In various embodiments, the one or more inserts 17 may be provided to a device user in a sterilized state, such that potentially harmful toxins, bacteria, and/or other harmful substances are prevented from contacting a needle prior to insertion into a patient's skin. The one or more inserts 17 may be at least substantially cylindrical in shape, having a hollow center and open ends. In various embodiments, the one or more inserts 17 may have an engagement feature on an exterior surface of the insert 17 configured to engage a corresponding feature on the freezable device 10, such that the insert 17 may remain operatively connected at least partially within the passage 15 of the freezable device 10 during use. The inserts 17 may comprise a disposable plastic and/or metal material configured for providing a sterile surface within one of the one or more passages 15 in the freezable device 10.

FIGS. 5A-5B illustrate bottom views of various alternative freezable devices 20, 30, 40 according to various embodiments. Referring initially to FIG. 5A, the freezable device 20 may define a “Y” shape, such that the passage 25 is defined as a notch in a side of the freezable device 20. In such embodiments, the passage surface may be defined as a portion of the perimeter surface extending around the perimeter of the freezable device 20. Moreover, as discussed above in reference to freezable device 10, the passage 25 may extend through the entirety of the freezable device 20 through a first surface (not shown) and a second surface 23 opposite the first surface. Moreover, at least one of the first surface and the second surface 23 may have a plurality of protrusions 26 distributed thereon.

Referring now to FIG. 5B, a freezable device 30 may have a substantially rectangular shape, although the freezable device 30 may have a variety of other shapes (e.g., square, round, elliptical, and/or the like). As illustrated in FIG. 5B, the freezable device 30 may define a continuous shape, such that no passage extends therethrough. Moreover, as illustrated, at least one of a first surface (not shown) and a second surface 33 opposite the first surface may have a plurality of protrusions 36 distributed thereon.

In reference to FIG. 5C, a freezable device 40 may have a plurality of passages 45 extending therethrough. The freezable device 40 may define a plurality of protrusions 46 distributed around the plurality of passages 45 on the second surface 43. Particularly in configurations in which the freezable device 40 is sterile, the freezable device 40 may be used for multiple needle pricks (e.g., multiple injections and/or blood draws) from a patient, which utilizing a previously unused passage 45 for each needle prick. Thus, each needle may be inserted into the patient through an otherwise unused passage 45.

Method of Use

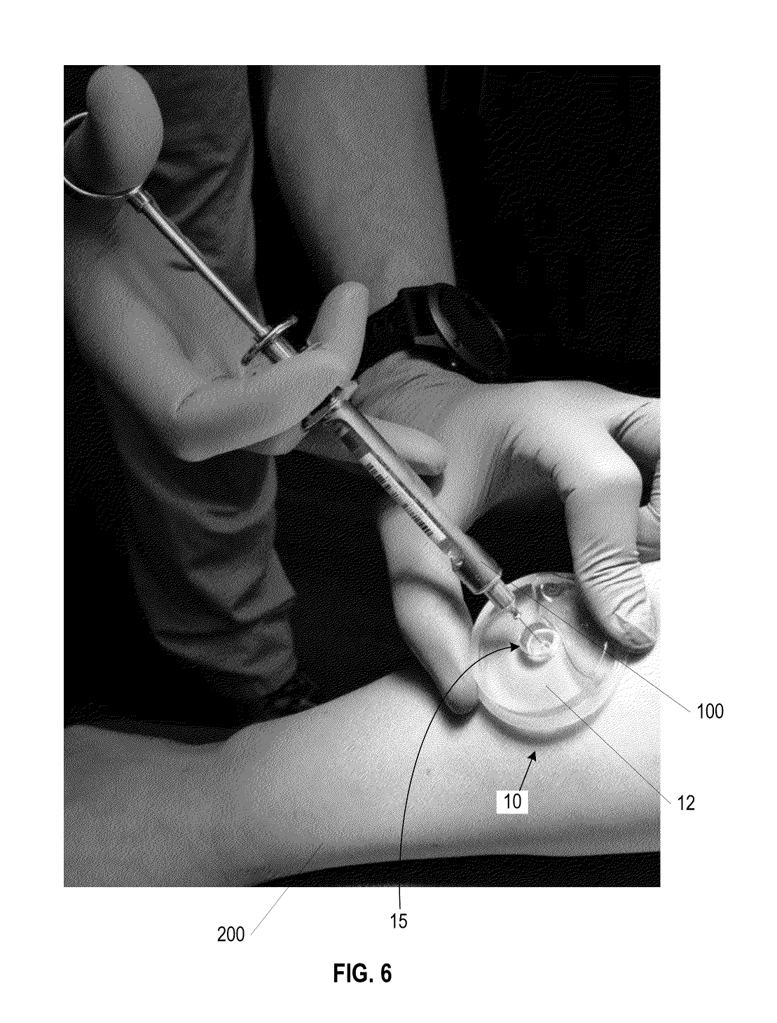

FIG. 6 illustrates a freezable device 10 in use. In various embodiments, the freezable device 10 may be cooled to freeze or otherwise chill the freezable substance or gel located within the interior cavity of the shell 11. For example, the freezable device 10 may be stored within a residential, commercial, and/or medical-grade freezer prior to use. After removal from the freezer, in various embodiments, the insert 17 may be inserted into the passage 15 of the freezable device 10.

The cooled freezable device 10 may be placed against the skin of a patient 200 such that at least a portion of the passage 15 is aligned with the needle insertion site at which a needle 100 is intended to be inserted. The cooled freezable device 10 is placed against the patient's skin such that at least some of the plurality of protrusions are in contact with the patient's skin. The freezable device 10 is depressed against the skin of the patient 200 such that the protrusions form corresponding indentions in the skin of the patient.

While the freezable device 10 is pressed against the skin of the patient 200, the needle 100 is inserted through the passage 15 and into the patient 200. In use, the freezable device 10 reduces perceived pain resulting from the needle insertion by providing a chilled surface at least partially around the needle insertion site, as well as providing a plurality of high pressure contact points by the plurality of protrusions being depressed into the surface of the patient's skin. The sensations perceived by the patients that are attributable to the cold-temperature of the freezable device 10 and the pressure points of the protrusions provide a localized sensory confusion sensation in the area surrounding the needle insertion site. Such sensory confusion may impede local pain receptors in the area immediately surrounding the needle insertion from providing a painful sensation felt by the patient.

### CONCLUSION

Many modifications and other embodiments of the inventions set forth herein will come to mind to one skilled in the art to which these inventions pertain having the benefit of the teachings presented in the foregoing descriptions and the following claims. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

For example, various embodiments of freezable device 10 may be sterile and provided for use by medical professionals. Such sterilized freezable device 10 may be disposable and intended for a single use prior to disposal. Alternatively, the freezable device 10 may be reusable and intended for residential use. As non-limiting examples, such a reusable freezable device 10 may be useful in providing insulin shots, injectable pain medication, and/or for other home-administered injections.

That which is claimed:

1. A freezable device for reducing pain perception during insertion of a needle into a patient, comprising:
a shell having a first surface and a second surface opposite the first surface; and
at least one passage extending through the device from the first surface to the second surface, wherein the passage is configured to at least partially surround a needle insertion site on the patient;
wherein at least one of the first surface and second surface comprise a plurality of protrusions configured to be pressed against the patient; and
wherein the shell defines an interior cavity, the inner cavity being filled, at least in part, with a first substance.

2. The freezable device of claim 1, wherein the first substance is a gel.

3. The freezable device of claim 1, wherein the shell comprises a pliable material.

4. The freezable device of claim 1, wherein the plurality of protrusions have a conical shape.

5. The freezable device of claim 1, wherein the plurality of protrusions have a pyramid shape.

6. The freezable device of claim 1, wherein the shell is sterile.

7. The freezable device of claim 1, wherein the shell comprises a perimeter surface surrounding the shell and extending between the first surface and the second surface to define the interior cavity of the shell; and
wherein the passage defines a passage surface at least partially surrounding the passage and extending between the first surface and the second surface; and
wherein the perimeter surface comprises the passage surface.

8. The freezable device of claim 1, wherein the passage defines a first hole extending through the first surface and a second hole extending through the second surface, and a passage perimeter surface extending between the first hole and the second hole.

9. The freezable device of claim 8, additionally comprising an insert to be placed within the passage, the insert being a hollow cylinder having open ends.

10. The freezable device of claim 9, wherein the insert is sterile.

11. The freezable device of claim 1, comprising two or more passages extending through the device from the first surface to the second surface.

12. A freezable device for reducing pain perception during insertion of a needle into a patient, comprising:
a shell having a first surface and a second surface opposite the first surface; and
at least one passage extending through the device from the first surface to the second surface, wherein the passage is configured to at least partially surround a needle insertion site on the patient and wherein a membrane covers at least one end of the passage;
wherein at least one of the first surface and second surface comprise a plurality of protrusions configured to be pressed against the patient; and
wherein the shell defines an interior cavity, the inner cavity being filled, at least in part, with a first substance.

13. The freezable device of claim 12, wherein the shell and the membrane are sterile.

14. The freezable device of claim 12, wherein the membrane is configured to be punctured by a needle.

15. The freezable device of claim 12, comprising two or more passages extending through the device from the first surface to the second surface, wherein at least one end of each of the two or more passages is covered by a corresponding membrane.

16. A method of reducing perceived pain during a needle insertion into a patient, the method comprising steps for:
reducing the temperature of a freezable device, the freezable device comprising a first surface and a second surface opposite the first surface, and a passage extending through the freezable device between the first surface and the second surface, and wherein at least one of the first surface and the second surface comprise a plurality of protrusions;
pressing the plurality of protrusions against the patient's skin such that a needle may be inserted through the passage and into the patient's skin at a needle insertion location; and
inserting the needle through the passage and into the patient's skin at the needle insertion location.

17. The method of reducing perceived pain during a needle insertion of claim 11, further comprising steps for sterilizing the freezable device prior to pressing the freezable device against the patient's skin.

18. The method of reducing perceived pain during a needle insertion of claim 11, further comprising steps for placing an insert into the passage prior to pressing the freezable device against the patient's skin.

19. The method of claim 11, further comprising steps for sterilizing the insert prior to inserting the insert into the passage.

20. The method of claim 11, wherein a membrane covers at least one end of the passage, and wherein inserting the needle through the passage and into the patient's skin further comprises puncturing the membrane.
